(19) United States
(12) Patent Application Publication
(10) Pub. No.: US 2016/0319214 A1
(43) Pub. Date: Nov. 3, 2016

(54) Oil, Lubricant Composition Having Improved Lubricity and Increased Coefficient of Performance, Uses and Mechanical Equipment

(71) Applicant: WHIRLPOOL S.A., São Paulo (BR)

(72) Inventors: Expedito Raimundo Pereira DOS SANTOS, Campinas (BR); Rosangela Maria MACHADO, Joinville (BR)

(21) Appl. No.: 15/105,754

(22) PCT Filed: Dec. 22, 2014
(86) PCT No.: PCT/BR2014/050052

§ 371 Date: Jun. 17, 2016

(30) Foreign Application Priority Data
Dec. 20, 2013 (BR) BR102013032982-7

(51) Int. Cl. C10M 169/04; C07C 15/107; C09K 5/04; C10M 105/06

(52) U.S. Cl. CPC C10M 169/04; C10M 105/06; C07C 15/107; C09K 5/042; C10N 2230/10; C10N 2240/30; C10M 2203/065; C10M 2223/041; C10N 2230/06; C09K 2205/12

(57) ABSTRACT

The present invention discloses an oil including at least one alkyl aromatic compound wherein the oil viscosity is lower than 3.0 cSt at the temperature of 40° C. The oil of the present invention shows a lower viscosity variation as a function of the temperature, what contributes to reduce the cooling system equipment wear and increase the equipment operation lifetime, for example, the operation lifetime of the cooling compressors. It also an object of the present invention a composition including said oil in combination with at least one fluid of the hydrocarbon (HC) type and its uses in mechanical equipments and mechanical equipments made with said oil and/or composition.

### FIELD OF THE INVENTION

The present invention relates to an oil having improved lubricity and increased coefficient of performance, to a lubricant composition and its uses, and to a cooling machine. More specifically, the oil of the invention comprises an alkyl aromatic compound and is useful, among other applications, in the preparation of lubricant compositions and in high performance improved cooling machines.

### PRIOR ART

The traditional cooling fluids applied in household cooling machines until mid 90's were chlorofluorocarbons (CFCs)-based compounds. However, when the use of said compounds was proven to damage the high atmosphere ozone layer, said compounds had their use limited and regulated under the terms of the Montreal protocol. The CFCs used in household applications were initially replaced with hydrofluorocarbons (HFCs) which have no ozone depletion potential (ODP). Nevertheless, the application of HFC compounds has a significant global warming potential (GWP), the reason why they have been replaced with hydrocarbons (HC)-based cooling fluids, especially in European and Asian domestic marketplaces.

The cooling fluids are used in cooling systems, together with a lubricating oil, to absorb heat and keep the environment cooled. In a cooling system, the compressor suctions the cooling fluid from the evaporator, thus reducing the pressure in this component. The fluid is then compressed by the compressor and travels to the condenser. In the condenser, the cooling fluid under high pressure releases heat to the environment and becomes liquid. The next component of the circuit is the control element that can be a capillary tube or an expansion valve. The control element reduced the pressure of the liquid cooling agent formed in the condenser and this pressure reduction allows the cooling agent to evaporate, thus returning to gas state when passing through the evaporator.

While the cooling fluid changes from liquid to gas state, it removes heat from the inside of the cooling system by means of the evaporator. The condenser releases heat to the environment outside the cooling system. The control element resists to the circulation of the cooling fluid, separating the high pressure side (condenser) from the low pressure side (evaporator).

So that they can perform such a function, the lubricating oil and the cooling fluid need to be miscible with each other in order to assure that the composition properly flows through the cooling circuit, thus avoiding the accumulation in different regions of the circuit to avoid the equipment wear.

The main purpose of the lubricating oil in cooling compressors is to lubricate mechanical contacts in the compressor by being an oil mixture that is diluted by the cooling fluid inside the compressor inner cavity. In addition to be compatible with the cooling fluid, the lubricating oil shall have a good oxidation resistance, a wide temperature range appropriate for the operation, shall be hydrolytically stable, have low mechanical resistance, excellent lubricity, among other properties.

Changing cooling fluids in cooling systems also causes a change of the lubricating oils used in said cooling systems, due to the need of adaptation between said components in the compressor of said cooling systems, so as to avoid, for example, reactions that produce acids in these systems and other components that impair the integrity of said system and its efficiency.

By searching the prior art in scientific and patent literatures, several documents were found referring to this subject matter. Those regarded as the most useful within the context of the present invention are listed below:

Document U.S. Pat. No. 6,248,256 discloses compositions for use in compression-cooling comprising (A) one cooling agent containing hydrocarbon C1-C8 and (B) an lubrication base oil formed by a hydrocarbon compound wherein the degree of unsaturation of the non-aromatic group is no more than 10% and in which the kinematic viscosity is no less than 5 mm²/s. Document U.S. Pat. No. 6,248,256 discloses in column 1, lines 45 to 52, that the strong reduction of the viscosity renders the lubrication performance insufficient to reduce abrasion, which could prevent the consistent use of the oil in coolers for long periods of time. Additionally, document U.S. Pat. No. 6,248,256 discloses in column 3, lines 52 to 57, that the lubricating oil kinematic viscosity shall not be lower than 5 mm²/s, as the sealing effect of the cooler or the lubricant performance would be impaired. Thus, document U.S. Pat. No. 6,248,256 has problems that would discourage those skilled in the art to produce an oil with a kinematic viscosity lower than 5 mm²/s for use in cooling machines. Therefore, document U.S. Pat. No. 6,248,256 would discourage those skilled in the art to reach the purpose claimed in the present invention.

Document BRPI0502759 of the present applicant is regarded as the one that comes closer to the present invention, although it does not anticipate or even suggests any of its features. It discloses a lubricating oil containing at least 80 wt % of alkylbenzene having a molecular weight of 120 to 288 and a viscosity from about 3.0 to 7.0 cSt at a temperature of 40° C. Additionally, it discloses a composition comprising the lubricating oil and even about 8 wt % of one or more additives. However, document BRPI0502759 discloses only lubricating oils or compositions containing alkylbenzenes having higher molecular weights—different from the alkyl aromatic compounds defined in the present invention, and that further show improved properties when compared to those of said document.

Document JP1982177097 discloses an oil composition for coolers that contain alkylbenzene having a specific dynamic viscosity as the main component, thus creating a good abrasion resistance, high stability with a cooling agent and a reduction of electric power consumption. The composition of document JP1982177097 uses an alkylbenzene having a dynamic viscosity from 5-20 cSt at 40° C. together with an additive consisting of hydrochloric acid acceptors, anti-abrasion agents, antioxidants and anti-forming agents. Document JP1982177097 differs from the present invention in that it uses alkyl aromatic compounds with heavier alkyl groups showing a higher viscosity than the oils of the present invention.

Document U.S. Pat. No. 6,207,071 discloses the use, in combination with the cooling agents HFC-134a and/or HFC-125, of a lubricating oil comprising an alkylbenzene oil containing 60 wt % of alkylbenzene having a molecular weight from 200 to 350 and additionally comprising 0.01 to 5.0 wt % of additives of a phosphoric ester. Document U.S. Pat. No. 6,207,071 differs from the present invention in that it uses HFC (hydrofluorocarbon) cooling agents that significantly contribute to aggravate the greenhouse effect (GWP) as mentioned above. Additionally, the composition described in U.S. Pat. No. 6,207,071 is not applicable under temperature conditions below about 20° C., as said lubricating oil shows low miscibility with the cooling agents HFC-134a and/or HFC-125 at temperatures below 20° C. That is, at temperatures about 0° C. or lower, said composition would not be feasible as the lubricating oil and the fluids HFC-134a and/or HFC-125 would not be miscible. As a result, said composition would not be indicated for application in cooling systems that would be subject to temperatures lower or equal to 0° C.

Document EP1018538 discloses a lubricating oil for cooling machines working as cooling agents consisting of at least one hydrocarbon such as HC-290 (propane) or HC-600a (isobutane) comprising a straight or branched chain alkylbenzene in which the molecular weight is within 200 and 350 g/mol, having 1 to 4 alkyl groups, each one comprising 1 to 19 carbon atoms, the total number of carbons in the alkyl group ranging from 9 to 15. According to EP1018538, as it can be seen in paragraph [0030], the alkylbenzenes containing branched alkyl groups are preferred due to the viscosity properties and behavior of the alkylbenzenes. Additionally, document EP1018538 does not provide specific examples with straight chain alkylbenzene oils as those claimed in the present invention. Thus, a person skilled in the art would not be encouraged to obtain the lubricating oils having the improved properties of the present invention since EP1018538 only mentions a wide variety of compounds without showing tests or giving suggestions that show the effectiveness of the oils of the present invention and, further, EP1018538 suggests that the alkylbenzenes containing branched alkyl groups are preferred, thus more effectively distinguishing from the object matter claimed in the present application.

New oils and new lubricating oil compositions in combination with cooling fluids showing improved properties to improve the efficiency of the compressors operation in cooling systems is constantly sought. However, from what is understood from the searched literature, documents anticipating or suggesting the taughts of the present invention have not been found, so that the solution proposed herein, as seen by the inventors, shows novelty and inventive activity when compared to the prior art.

### SUMMARY OF THE INVENTION

One of the objects of the present invention is to provide an oil comprising at least one alkyl aromatic compound and having improved physicochemical properties, able to provide a stable lubricating film, suitably lubricating the components of a mechanical equipment with as low viscosity as possible, therefore, increasing the performance of said equipment without damaging its parts.

Another object of the present invention is a lubricant composition comprising said oil and one cooling fluid containing at least one component of a HC (hydrocarbon) group.

It is another object of the invention to use of the oil and/or the lubricant composition in cooling machines, as well as the improved cooling machines comprising the use of said substances.

The concept of the invention common to the several objects of the invention is to improve the lubricity conditions and to maintain the miscibility unchanged even at low temperatures, avoiding the collapse of machines (from the collapse of the lubricating oil film) using lubricating fluids, and maintaining high reliability for a long period of time, at least during the thermal machines lifetime, such as cooling compressors. Additionally, the coefficient of performance (COP) in thermal machines, such as compressors, was increased. It was obtained from the development of an oil comprising at least one alkyl aromatic compound wherein the oil viscosity is lower than 3.0 cSt at the temperature of 40° C.

The oil of the present invention not only shows a low viscosity at 40° C. (<3.0 cSt), but also a lower viscosity variation as a function of the temperature, so that it contributes to reduce the wear of the cooling system equipment and to increase the equipment operation lifetime, such as the operation lifetime of the cooling compressors, as the formation of a stable oil film during the operation of the mechanical equipment, such as a compressor.

In a first aspect, therefore, the present invention provides an oil comprising at least one alkyl aromatic compound wherein the oil viscosity is lower than 3.0 cSt at the temperature of 40° C.

In one embodiment of the present invention it provides an oil comprising at least one alkyl aromatic compound of the formula (I):

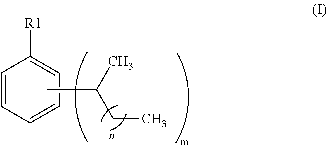

wherein:

when R1 is H, (n) is an integer from 1 to 7 and (m) is an integer from 1 to 3;

when R1 is methyl, ethyl or isopropyl, (n) is an integer from 1 to 7 and (m) is an integer from 1 to 2; and wherein the oil viscosity is lower than 3.0 cSt at the temperature of 40° C.

In one embodiment of the present invention, said oil comprises a viscosity from 1.0 to 3.0 cSt at the temperature of 40° C. In one embodiment the oil viscosity is between 1.0 and below 2.5 cSt at the temperature of 40° C. In one embodiment the oil viscosity is between 1.0 and below 2.2 cSt at the temperature of 40° C. In one embodiment of the present invention, said oil comprises a total amount of hydrocarbon radicals bonded to the aromatic ring within 1 and 12. In one embodiment of the present invention, said oil comprises at least 80% by mass of the at least one alkyl aromatic compound. In another embodiment of the present invention, the oil of the present invention additionally comprises at least one additive selected from the group consisting of oxidation resistance, thermal stability promoters, corrosion inhibitors, metal deactivators, lubricity additives, viscosity index enhancers, fluidity lowering agents, floc point lowering agents, detergents, dispersants, foaming agents, anti-wear agents and high-pressure resistance agents. In another embodiment of the oil of the present invention, the alkyl aromatic compound comprises a molecular weight between 134 and 218. In one preferred embodiment of the present invention, the oil is used as a lubricating oil. In one preferred embodiment of the present invention the alkyl aromatic compound is a linear alkyl aromatic compound.

Another object of the present invention is a lubricant composition having improved lubricity and increased coefficient of performance when compared to those oils common in the art comprising said oil above defined in combination with at least one cooling fluid from the HC (hydrocarbon) group.

In one embodiment of the lubricant composition of the present invention, the cooling fluid from the HC group is HC-600a, HC-290 or combinations thereof.

It is another object of the present invention to use of the oil or the composition of the invention to manufacture mechanical equipment using said substances for lubrication.

In one embodiment, said use is in cooling machines. In one embodiment, the cooling machine is a compressor or a hermetic compressor.

It is further another object of the present invention a mechanical equipment comprising, an alkylaromatic oil, as defined above or a lubricant composition, as defined above.

In one embodiment, the mechanical equipment is a compressor, hermetic compressor or cooling machine.

### FIGURES DESCRIPTION

FIG. 1 provides a comparative viscosity (cP) per temperature (° C.) of different oil herein indicated. This figure also provides a clear indication of the lower viscosity variation of the oils of present invention compared to the oils of the state of the art.

FIG. 2 provides a viscosity (cP) per temperature (° C.) of sample 0 oil.

FIG. 3 provides a viscosity (cSt) per temperature (° C.) of sample 0 oil.

FIG. 4 provides a solubility curve of both Linear AlkylBenzene with mean molecular weight of 240 g/mol (LAB240) and Linear AlkylBenzene with mean molecular weight of 190 g/mol (LAB190—Sample 0) within the invention with a R600a refrigerant. Provides a clear indication the LAB190 oil contains lower equalized pressure for the same room temperature.

FIG. 5 provides an oxidation accelerated test in an autoclave, where it can be noticed that there is no significant difference between a Linear AlkylBenzene with mean molecular weight of 240 g/mol (LAB ISO5) and Linear AlkylBenzene with mean molecular weight of 190 g/mol (LAB ISO2—Sample 0) between 25 and 175° C.

FIG. 6 provides an oxidation stability test performed in a DSC 204 HP Phoenix. Test Condition: Temp. programed: 30-300° C.; heating hate: 5K/min; pressure 35 Bar; flow of oxygen: 100 mL/min. It can be seen that the oils of the present invention (LAB190—Sample 0) shows a higher resistance against oxygen and begin the oxidation process in a temperature higher than the other oils of the state of the art.

FIG. 7 provides an accelerated life testing for oil degradation (qualitative analysis) at a compressor pumping air (opened circuit/5 bars) of the Embraco EM family of compressors. Shows that the oils within the scope as defined by the present invention (LAB190—Sample 0) presents a higher resistance against oxidation the the oils of the state of the art. It was not observed impact on the dieletric strength breaking down.

FIG. 8 and FIG. 9 provides an accelerated life testing for oil degradation at a compressor pumping air (opened circuit/5 bars) of the Embraco EM family of compressors. Shows that the oils within the scope as defined by the present invention (LAB190—Sample 0) presents bigger resistance against oxidation the the oils of the state of the art. It was not observed impact on the dieletric strength breaking down.

FIG. 10 provides an Infrared Spectometry Analysis (FTIR) of sample 0 oil.

### DETAILED DESCRIPTION OF THE INVENTION

The concept of the invention common to the several objects of the invention is to improve the lubricity conditions and to maintain the miscibility unchanged even at low temperatures, avoiding the collapse of machines using lubricating fluids, and maintaining high reliability for a long period of time, at least during the thermal machines lifetime, such as thermal machines, such as cooling compressors. Additionally, the coefficient of performance (COP) in thermal machines, such as compressors, was increased. It was obtained from the use of an oil comprising at least one alkyl aromatic compound wherein the oil viscosity is lower than 3.0 cSt at the temperature of 40° C.

The oil of the present invention not only shows a low viscosity at 40° C. (<3.0 cSt), but also a lower viscosity variation as a function of the temperature, so that it contributes to reduce the wear of the cooling system equipment and to increase the equipment operation lifetime, such as the operation lifetime of the cooling compressors.

Thus, the present invention shows an oil having improved properties comprising at least one alkyl aromatic compound wherein the oil viscosity is lower than 3.0 cSt at the temperature of 40° C.

In an embodiment the at least one alkyl aromatic compound is of the formula (I)

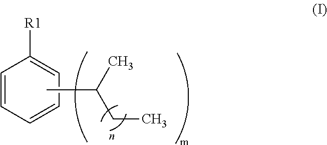

wherein when R1 is H, (n) is an integer from 1 to 7 and (m) is an integer from 1 to 3; when R1 is methyl, ethyl or isopropyl, (n) is an integer from 1 to 7 and (m) is an integer from 1 to 2;

In an embodiment of the present invention, the oil comprises one alkyl aromatic compound having a molecular weight between 134 and 218.

In an embodiment of the present invention, the oil will be described for the application with heat transfer organic cooling agents including at least a constituent of a hydrocarbons (HC) group, such as cooling fluids HC-600a, HC-290 or combinations thereof, for use in cooling machines, such as cooling compressors used in cooling systems, particularly for household use. Said cooling compressors can be, for example, hermetic compressors.

Considering the use of said oil together with cooling fluids containing hydrocarbon (HC), such as HC-600a and/or HC-290, it is desirable that the lubricating oil shows a viscosity lower than 3.0 cSt at 40° C., such as within the range of about 1.0 to about 2.9 cSt, possibly between 1.0 and below 2.5 cSt, possibly between 1.0 and below 2.2 cSt, at the temperature of 40° C. Additionally, the oil of the present invention shows a lower viscosity variation as a function of the temperature, so that it contributes to reduce the wear of the cooling system equipment and to increase the equipment operation lifetime, such as, the operation lifetime of the cooling compressors.

In view of the improvement of the property that impedes the collapse of hermetic compressor during a long-term operation, the lubricating oil is selected from those containing at least 80%, more preferably at least 85% and more preferably at least 90% of the alkyl aromatic compound of the present invention.

Examples of alkyl group containing from 4 to 12 carbon atoms are butyl (including all isomers), pentyl (including all isomers), hexyl (including all isomers), heptyl (including all isomers), octyl (including all isomers), nonyl (including all isomers), decyl (including all isomers), undecyl (including all isomers), dodecyl (including all isomers).

In view of the stability and viscosity of the alkyl aromatic compounds, the straight chain monoalkyl groups are preferred, since the chain linearity leads to a better lubricity (with reduced viscosity) and the presence of an alkyl group positively influences the chemical stability of an alkyl aromatic oil.

These alkyl groups can be of a straight or branched chain. However, in view of the alkylbenzenes stability and viscosity, the straight chain monoalkyl groups are preferred, since the chain linearity leads to a better lubricity and the presence of an alkyl group positively influences the chemical stability of the alkylbenzene oil.

The number of alkyl groups in the abovementioned benzene is defined as being from 1 to 4. However, in view of the stability and availability of the alkylbenzene, it is more preferable to select an alkylbenzene containing one or two alkyl groups, that is, one monoalkylbenzene, one dialkylbenzene or a mixture thereof, more preferably, one monoalkylbenzene in a straight or branched chain.

The alkyl aromatic compounds of the present invention are preferably produced from an alkylation process of aromatic compounds with alpha-olefins in the presence of a proper catalyst.

The aromatic compounds that can be used as a raw material for the synthesis of the alkyl aromatic compounds include benzene, toluene, ethylbenzene, cumene, among others.

The alkylating agents that can be used when producing the alkyl aromatic compounds include straight chain alpha-olefin containing from 4 to 12 carbon atoms, more preferably from 4 to 10 carbon atoms.

A catalyst to be used in the homogeneous alkylation process include, for example, a Friedel-Crafts catalyst, such as aluminum chloride or zinc chloride; or an acid catalyst defined by sulfuric acid, phosphoric acid, hydrofluoric acid and by the heterogeneous alkylation by the use of supported solid catalysts and activated clay, such as zeolites among others.

Under some usage conditions, the alkyl aromatic compounds with a kinematic viscosity preferably from 1.3 to about 2.9 cSt, possibly between 1.3 and below 2.5 cSt, possibly between 1.3 and below 2.2 cSt, at the temperature of 40° C. as described herein, satisfactorily work as complete lubricants. However, the lubricating oil may additionally contain other materials, usually called additives.

In an embodiment, the oil of the present invention may comprise at least one additive. Among the additives used, the following can be mentioned: oxidation resistance enhancers and thermal stability enhancers, corrosion inhibitors, metal deactivators, lubricity additives, viscosity index enhancers, fluidity lowering agents and floc point lowering agents, detergents, dispersants, antifoaming agents, anti-wear agents and extreme pressure resistant additives. Many additives are multifunctional. For example, certain additives may have both extreme pressure resistance and anti-wear properties, or both functions as a metal deactivator and a corrosion inhibitor. Additionally, all additives in a composition shall not exceed, preferably, 8 wt % or, more preferably, 5 wt % of the oil total formulation.

An effective amount of the preceding additive types is usually within the range from 0.01 to 5% for an antioxidant component, from 0.01 to 5% for a corrosion inhibitor component, from 0.001 to 0.5% for a metal deactivator component, from 0.5 to 5% for the lubricity additives, from 0.01 to 2% for each viscosity index enhancer and fluidity and/or floc point lowering agents, from 0.1 to 5% for each detergent and dispersant, from 0.001 to 1% of antifoaming agents, and from 0.1 to 3% for each extreme pressure resistant component and anti-wear component. All these percentages are by weight and based on the oil composition total. Nevertheless, it should be understood that higher or lower amounts of additives may be used as a function of particular aspects of the oil and its application, and that a type of simple molecule or a type of mixture can be used for each type of additive. Besides, the examples mentioned herein shall be understood as exemplifying, and not as limitative examples.

Examples of certain oxidation resistance enhancers and thermal stability enhancers are the diphenyl-dinaphthyl-, and phenylnaphthyl-amines, in which the phenyl and naphthyl groups can be replaced, that is, N,N′-diphenyl phenylenediamine, p-octyldiphenylamine, p,p-dioctyldiphenylamine, N-phenyl-1-naphthylamine, N-phenyl-2-naphthylamine, N-(p-dodecyl)phenyl-2-naphthylamine, di-1-naphthylamine, and di-2-naphthylamine; phenothiazines, such as N-alkylphenothiazines; imino(bisbenzyl); and phenols, such as 6-(t-butyl)phenol, 2,6-di-(t-butyl)phenol, 4-methyl-2,6-di-di-(t-butyl)phenol, 4,4′-methylenebis(-2,6-di-(t-butyl)phenol).

Examples of certain cuprous metal deactivators are imidazole, benzomidazole, 2-mercaptobenzothiazole, 2,5-di-mercaptothiazole, salicylidine-propylenediamine, pyrazole, benzotriazole, tolutriazole, 2-methylbenzamidazole, 3,5-dimethyl pyrazole, and methylene bisbenzotriazole. Benzotriazole derivatives are preferred. Other more common examples of metal deactivators and/or corrosion inhibitors include organic acids and esters thereof, metal salts, and anhydrides, that is, N-oleyl-sarcosine, sorbitan monooleate, plumb naphthenate, dodecenyl-succinic acid and esters thereof and partial amides, and 4-nonylphenoxy acetic acid; aliphatic and cycloaliphatic primary, secondary and tertiary amines and amine salts of organic and inorganic acids, that is, oil-soluble alkylammonium carboxylates; heterocyclic compounds containing nitrogen, that is, thiodiatriazols, substituted imidazolines, and oxazolines; barium dinonyl naphthalene sulfonate; quinolines, quinones, and anthraquinones; propyl galate; amide ester and derivatives of succinic alkenyl anhydrides or acids, dithiocarbamates, dithiophosphates; amine salts of alkyl phosphate acid and derivatives thereof.

Examples of certain lubricity additives include long chain fatty acids and natural oils derivatives, such as esters, amines, imidazolines, and borates.

Examples of certain viscosity index enhancers include polymethacrylates, vinylpyrrolidone copolymers and methacrylate, polybutenes, and styrene-acrylate copolymers.

Examples of certain fluidity and/or floc point lowering agents include polymethacrylates, such as methacrylate-ethylene-vinyl acetate thermopolymers; alkylated naphthalene derivatives; and Friedel-Crafts products catalyzed by the condensation of urea with naphthalenes or phenols.

Examples of certain detergents and/or dispersants include polybutenylsuccinic acid amides; polybutenyl phosphonic acids derivatives; long chain alkyl aromatic sulfonic acids and salts thereof; and metallic salts of alkyl sulfides, alkyl phenols, and of condensation products of alkyl phenols and aldehydes.

Examples of certain antifoaming agents include silicone and some acrylates.

Examples of certain extreme pressure resistant and anti-wear additives include sulfurized fatty acids and fatty acids esters, such as sulfurized octylthalate; sulfurized terpenes; sulfurized olefins; organopolysulfides; organophosphorus derivatives including amine phosphates, alkyl acid phosphates, dialkyl phosphates, aminedithiophosphate, trialkyl and triaryl phosphorothionates, trialkyl and triaryl phosphines, and dialkylphosphites, that is, amine salts of phosphoric acid monohexyl ester, amine salts of dinonylnaphthalene sulfonate, triphenyl phosphate, trinaphthyl phosphate, diphenyl cresyl and dicresyl phenyl phosphates, naphthyl diphenyl phosphate, triphenylphosphorothionate; dithiocarbamates, such as a antimony dialkyl dithiocarbamate; chlorinated hydrocarbons and/or fluorates and xanthalates.

In one embodiment of the present invention, aiming to improve the cooling mechanism for wear resistance and loading resistance, the lubricating oil additionally comprises up to about 8 wt % of one or more additive compounds from at least one type of phosphorus compound selected from the group consisting of phosphoric ester, phosphoric acid esters, amine salts of phosphoric acid esters, chlorophosphoric esters and phosphorus esters.

In one embodiment, the oil comprises from 0.01 to 5.0 wt % and, more particularly, from 0.005 to 5.0 wt % (based on the total amount of the oil composition) of an ester phosphate compound.

Phosphoric esters used herein include tributyl phosphate, triphenyl phosphate, trihexyl phosphate, triheptyl phosphate, trioctyl phosphate, trinonyl phosphate, tridecyl phosphate, tritetradecyl phosphate, tripentadecyl phosphate, trihexadecyl phosphate, triheptadecyl phosphate, trioctadecyl phosphate, trioleyl phosphate, triphenyl phosphate, tricresyl phosphate, trixylyl phosphate, cresyldiphenyl phosphate and xylydiphenyl phosphate.

Esters of phosphoric acid used herein include monobutyl phosphate acid, monopentyl phosphate acid, monohexyl phosphate acid, monoheptyl phosphate acid, monooctyl phosphate acid, monononyl phosphate acid, monodecyl phosphate acid, monoundecyl phosphate acid, monododecyl phosphate acid, monotridecyl phosphate acid, monotetradecyl phosphate acid, monopentadecyl phosphate acid, monohexadecyl phosphate acid, monoheptadecyl phosphate acid, monooctadecyl phosphate acid, monooleyl phosphate acid, dibutyl phosphate acid, diphenyl phosphate acid, dihexyl phosphate acid, diheptyl phosphate acid, dioctyl phosphate acid, dinonyl phosphate acid, didecyl phosphate acid, diundecyl phosphate acid, didodecyl phosphate acid, ditridecyl phosphate acid, ditetradecyl phosphate acid, dipentadecyl phosphate acid, dioctadecyl phosphate acid and dioleyl phosphate acid. Examples of ester amine salts of phosphoric acid are methylamine, ethylamine, propylamine, butylamine, pentylamine, hexylamine, heptylamine, octylamine, dimethylamine, diethylamine, dipropylamine, dibutylamine, dipentylamine, dihexylamine, diheptylamine, dioctylamine, trimethylamine, triethylamine, tripropylamine, tributylamine, tripentylamine, trihexylamine, triheptylamine, and trioctyl ester amine of phosphoric acid. Examples of chlorinated phosphoric ester are tris-dichloropropyl phosphate, tris-chloroethyl phosphate tris-chloropentyl phosphate, and polyoxyalkylene bis[(dichloroalkyl)] phosphate.

Examples of phosphorous esters are dibutyl phosphite, dipentyl phosphite, dihexyl phosphite, diheptyl phosphite, dioctyl phosphite, dinonyl phosphite, didecyl phosphite, diundecyl phosphite, didodecyl phosphite, dioleyl phosphite, diphenyl phosphite, dicresyl phosphite, tributyl phosphite, tripentyl phosphite, trihexyl phosphite, triheptyl phosphite, trioctyl phosphite, trinonyl phosphite, tridecyl phosphite, triundecyl phosphite, tridodecyl phosphite, trioleyl phosphite, triphenyl phosphite and tricresyl phosphite. It is also possible to use a mixture of said compounds.

These phosphorous compounds can be mixed with the lubricating oil within a desired mixture range. However, it is usually preferred to blend these phosphorous compounds within the range from 0.005 to 5.0 wt %, more preferably from 0.01 to 3.0 wt % based on the total amount of the lubricating oil composition for cooling purposes (a total of alkylbenzene oil of this invention and the total additives).

In a second aspect, the present invention discloses a lubricant composition comprising the oil of the present invention in combination with one cooling fluid. In an embodiment, said cooling fluid is at least one selected from the HC (hydrocarbon) group. In a more preferred embodiment, the fluid is selected from HC-600a, HC-290 or combinations thereof. In a still more preferred embodiment, the lubricant composition of the present invention is preferably used in cooling machines. Among the cooling machines, we can mention compressors, such as hermetic compressors.

In another aspect, the present invention defines the use of the oil for the manufacture of a mechanical equipment. In an embodiment, said mechanical equipment is a cooling machine. In a further embodiment, said cooling machine is a compressor. In a still further embodiment, said compressor is a hermetic compressor.

In another aspect, the present invention defines the use of the lubricant composition for the manufacture of cooling machines. In an embodiment of the present invention, said cooling machines are compressors or hermetic compressors.

One of the objects of the invention is the use of the oil and/or lubricant composition in cooling machines, as well as in the improved cooling machines comprising said substances.

In another aspect of the present invention, an improved cooling machine is disclosed containing the oil or a lubricant composition inside its inner cavity as defined in the present invention. In an embodiment, the cooling machine is a compressor or hermetic compressor.

Therefore, the objects of the present invention contribute to maintain the proper lubricity and miscibility conditions of the lubricating compositions even at low temperatures, thus avoiding the collapse of machines using it, and maintaining high reliability for a long period of time, at least during the machines lifetime, such as thermal machines, for example, cooling compressors. Additionally, the coefficient of performance (COP) in thermal machines, such as cooling compressors, was increased, thus resulting in energy efficiency improvement in the cooling cycle of machines using the oil and/or the lubricant composition of the present invention.

### EXAMPLES

### Embodiments

In order to perform tests proving the desired properties of the oils of the present invention, an oil comprising an alkylbenzene compound having a paraffinic side chain with 8 carbon atoms called “sample 0” and “Sample 1” was synthesized:

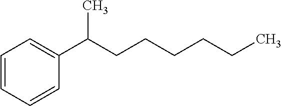

Additionally, an anti-wear (AW) additive was added to “Sample 0” and “Sample 1” oil defined as follows:

Chemical name: butylated triphenyl phosphate

Chemical formula: mixture

Chemical family: aryl phosphate

Amount: 2.0±0.3% (m/m) relative to the oil total mass

Substances contained in the additive
t-butylphenyl diphenyl phosphate
Bis (t-butylphenyl) phenyl phosphate
Tri (t-butylphenyl) phosphate
Triphenyl phosphate

The oil of the present invention mentioned above was compared to a commercial sample of LAB 240A (paraffinic side chain alkylbenzene having 10-13 carbon atoms, mean molecular weight of 238-245) and with other lubricating oils comprising alkylbenzene compound having a side chain with 10 carbons called “sample 2”:

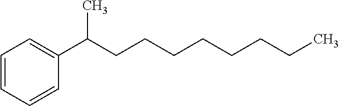 [SMILES: CCCCCCCCC(C)c1ccccc1]

The alkylbenzene compound of sample 2 was synthesized from benzene alkylation with an alkylating agent, in this case, from straight chain alpha-olefin containing 10 carbons (1-decene) by means of the homogeneous catalysis process using a Friedel-Crafts (aluminum chloride) catalyst or by means of a heterogeneous catalysis process using zeolites as catalysts (preferably dealuminated zeolites-HY).

The lubricating oil of the present invention was also compared to another lubricating oil comprising alkylbenzene compound having a side chain with 12 carbons called “sample 3”:

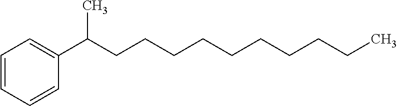 [SMILES: CCCCCCCCCCC(C)c1ccccc1]

The alkylbenzene compound of sample 3 was synthesized from benzene alkylation with an alkylating agent, in this case, from straight chain alpha-olefin containing 12 carbons (1-dodecene) by means of a homogeneous catalysis process using a Friedel-Crafts (aluminum chloride) catalyst or by means of a heterogeneous catalysis process using zeolites as catalysts (preferably dealuminated zeolites-HY).

Additionally, the butylated triphenyl phosphate additive as defined above was also added to samples 2 and 3.

Lubricating Oils Viscosity Analysis

The samples characteristics can be seen in table 1 below:

TABLE 1

| | LAB 240A | LAB 240A | | | | |
|---|---|---|---|---|---|---|
| | viscosity | viscosity | | | | |
| Physico- | grade | grade | | | | |
| chemical | specification | typical value | | | | |
| Properties | ISO 5 | ISO 5 | Sample 3 | Sample 2 | Sample 1 | Sample 0 |
| Color | Max. 1.0 | <0.5 | <0.5 | <0.5 | <0.5 | <0.5 |
| Density (g/cm³) | 0.845-0.870 | 0.860 | 0.850 | 0.860 | 0.859 | 0.861 |
| Viscosity 40° C., | 4.10-4.80 | 4.21 | 4.81 | 2.9 | 2.16 | 2.11 |
| (cSt) | | | | | | |
| Viscosity 100° C., | 1.25-1.60 | 1.33 | 1.53 | 1.34 | 1.18 | 0.91 |
| (cSt) | | | | | | |
| Miscibility | Max. −50 | −70 | −68 | −70 | −70 | −68 |
| HC-600a (° C.) | | | | | | |
| Flash point | Min. 135 | 142 | 144 | 126 | 112 | 114 |
| (° C.) | | | | | | |
| Combustion point | Min. 145 | 150 | 152 | 134 | 118 | N/A |
| (° C.) | | | | | | |
| Total acidity | Max. 0.03 | <0.01 | 0.03 | 0.01 | 0.02 | 0.024 |
| TAN(mg KOH/g) | | | | | | |
| Dieletric Strength, | N/A | N/A | N/A | N/A | N/A | 60 |
| Kv | | | | | | |
| Ester Phosphate | N/A | N/A | N/A | N/A | N/A | 2.08 |
| Content (BTP), % | | | | | | |
| mass | | | | | | |

As it can be seen from table 1, a significant viscosity reduction of sample 0 and 1 at 40° C. occurred.

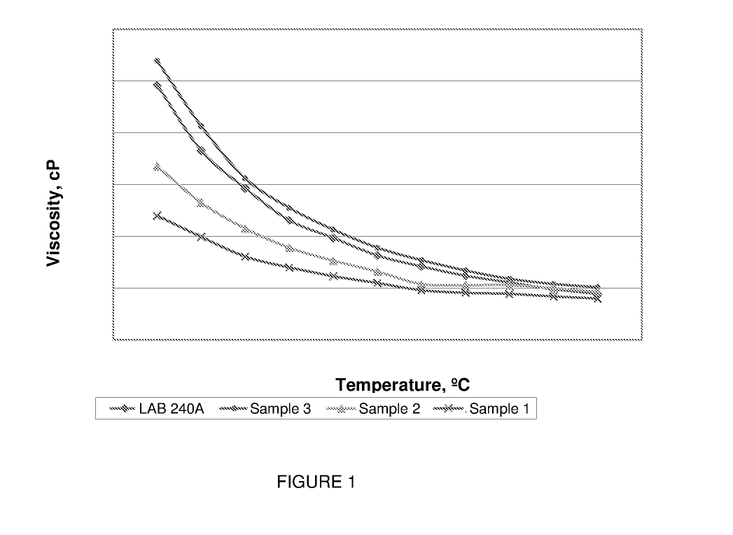

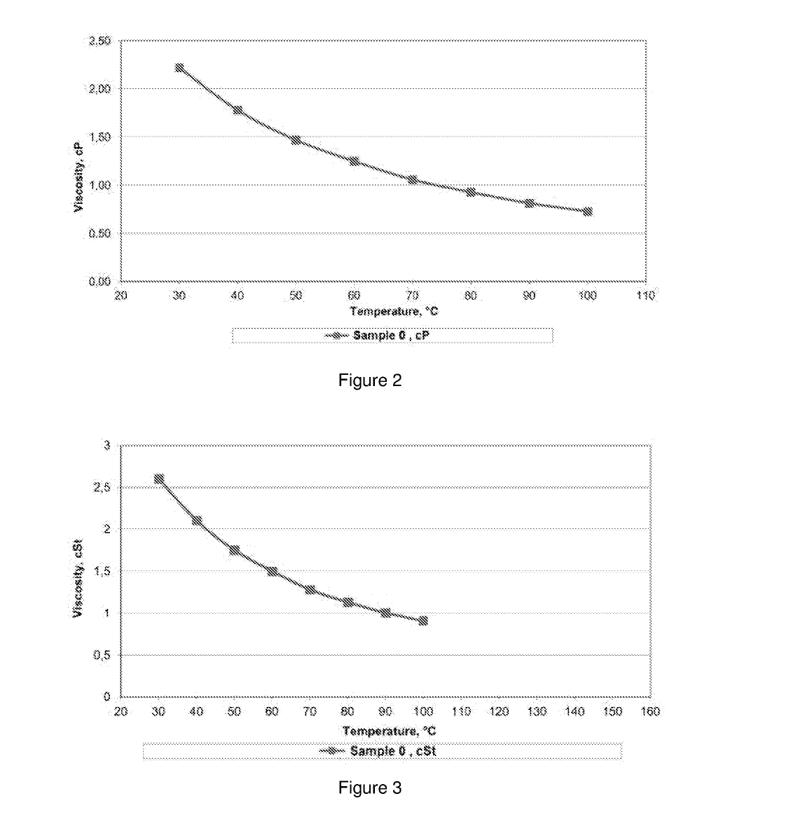

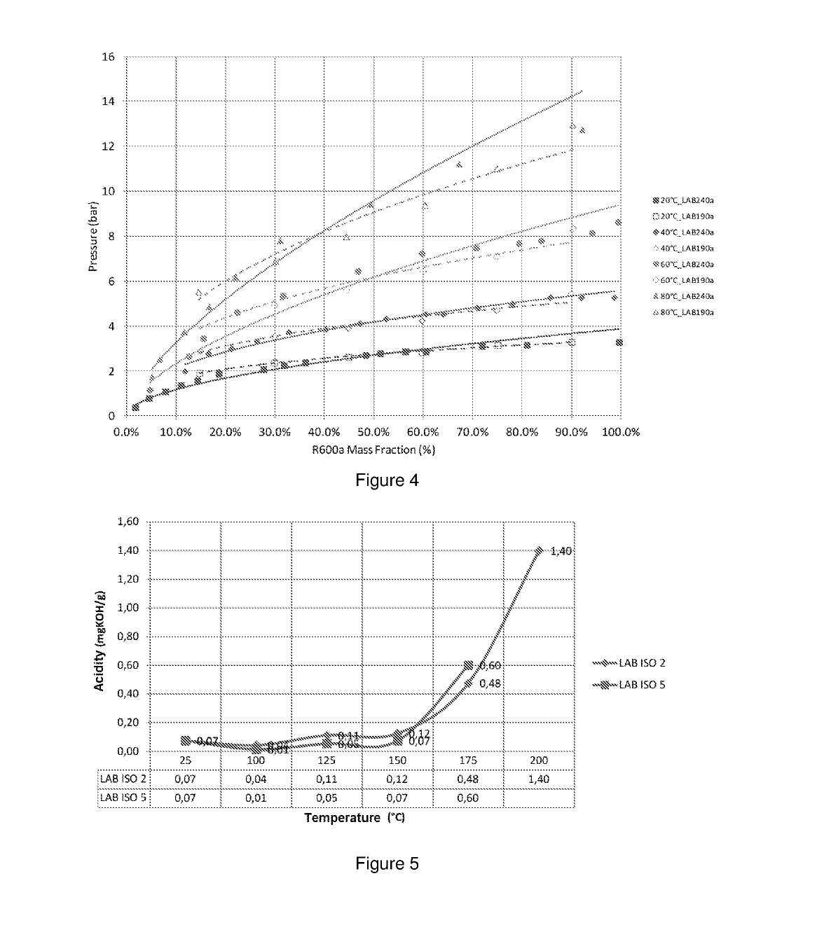

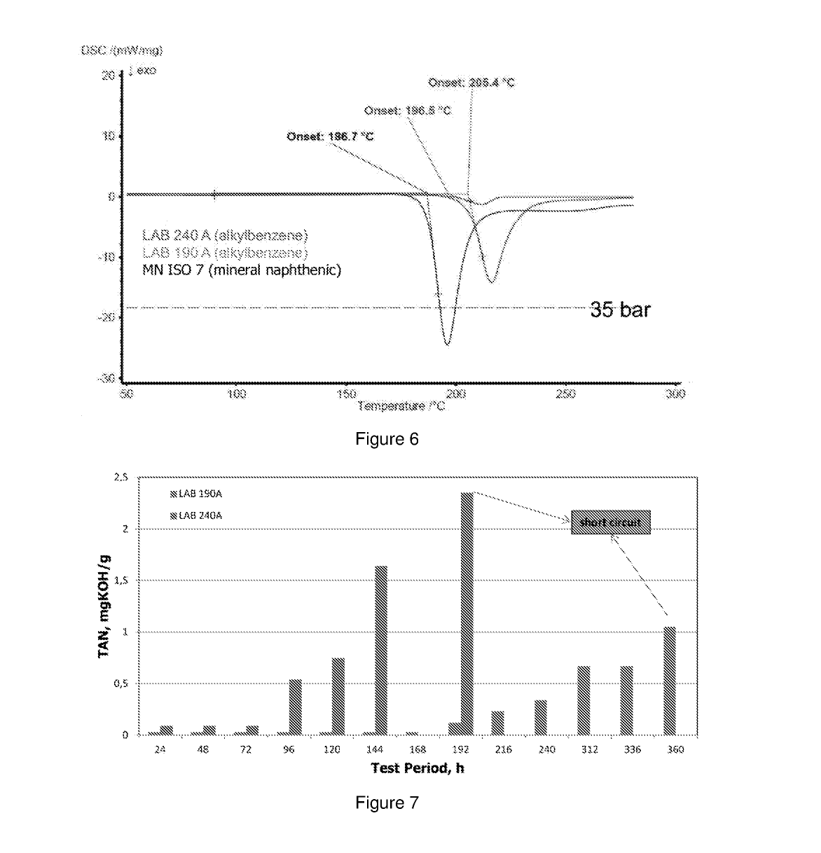

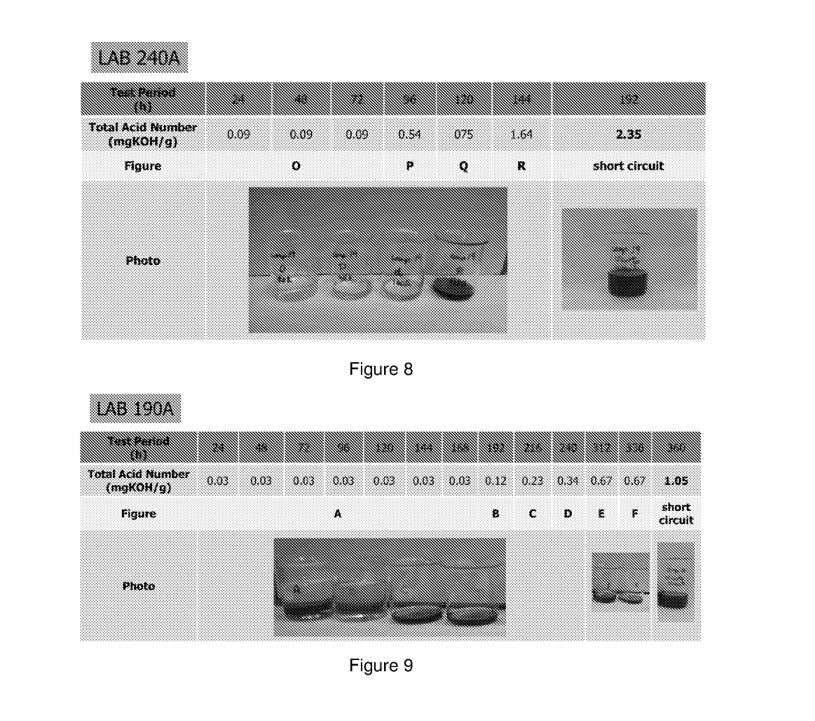

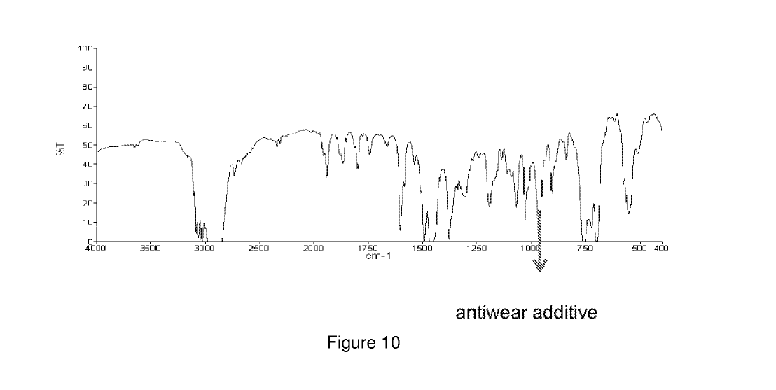

Additionally, FIG. 1 shows a comparison of the samples viscosities profile as a function of the temperature within the interval from 30° C. to 130° C. As it can be seen in FIG. 1, the sample 0 and 1 lubricating oil has a much lower viscosity variation with the temperature when compared to LAB 240A viscosity grade ISO 5 samples and to “sample 3”. Such a property contributes to reduce the cooling system equipment wear and to increase the equipment operation lifetime, such as the cooling compressors operation lifetime.

Chemical Compatibility Test with the Cooling Fluid

In order to analyze the compatibility of the lubricating oils of reference sample LAB 240A viscosity grade ISO 5 and “samples 0, 1, 2 and 3” with the hydrocarbon-based cooling fluids, sealed tube tests were carried out (Ashrae 97) using the cooling fluid HC-600a at 175° C. for 14 days. The tests results can be seen in table 2 below:

TABLE 2

| Analysis | Reference | Sample 3 | Sample 2 | Sample 1 | Sample 0 |
|---|---|---|---|---|---|
| Total Acidity, | Satisfactory | Satisfactory | Satisfactory | Satisfactory | Satisfactory |
| TAN mg | Result | Result | Result | Result | Result |
| KOH/g | | | | | |
| Lubricating oil/ | | | | | |
| cooling fluid | | | | | |
| change | | | | | |
| Copper plating | | | | | |
| Steel coupon | | | | | |
| change | | | | | |
| Copper | | | | | |
| coupon | | | | | |
| change | | | | | |

Composition Test in a Compressor During Operation

So as to prove the feasible use of the oils composition of the present invention, tests were carried out using a composition containing the “sample 1” oil in combination with the cooling fluid HC-600a. The tests results are summarized in table 3 below:

TABLE 3

| | Compressor | Test | Period | |
|---|---|---|---|---|
| LIMS | Model | Condition (° C.) | (h) | Results |
| 12/04922 | Model 1 | Lifetime test with | 2000 | Satisfactory |
| | | capillary tube | | |
| | | (−28/54/132) | | |
| | | Cyclic lifetime test | 2000 | Satisfactory |
| | | (−5/70/135) | | |
| 13/11173 | Model 2 | Wear test | 500 | Satisfactory |
| | | (−10/90) | | |
| | | On-Off test 125 K | 500 | Satisfactory |
| | | (−10/55) | | |

Potential Gains in Mechanical Loss

In order to prove the efficiency of the lubricating oil and the compositions of the present invention, the theoretical and experimental gains were calculated with sample 1 compared to the reference sample LAB 240A viscosity grade ISO 5. The results are showed in table 4 below:

TABLE 4
Sample 1
Optimization - Compressor Model 2

| | |
|---|---|
| Theoretical gain compared to | Experimental gain compared to |
| sample LAB 240A viscosity grade | sample LAB 240A viscosity grade |
| ISO 5 | ISO 5 |
| 1.0 W | 1.0 W |
| 2.8% in COP | 2.5% in COP |

As it can be seen from table 4, the tests showed expressive gains when using the composition containing the sample 1 lubricating oil. Gains of 1.0 W and 2.5% in the coefficient of performance (COP) experimentally obtained while applying the oils of the present invention in hermetic compressors during operation confirmed the efficiency of the oils of the present invention when compared to the prior art oils.

Those skilled in the art will appreciate the knowledge presented herein and will be able to reproduce the invention in the embodiments described and in other variations within the scope of the appended claims.

1. An oil characterized in that it comprises at least one alkyl aromatic compound wherein the oil viscosity is lower than 3.0 cSt at the temperature of 40° C.

2. An oil characterized in that it comprises at least one alkyl aromatic compound of the formula (I): 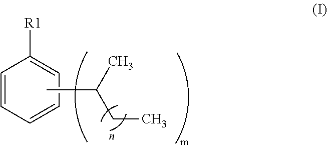
wherein:
when R1 is H, (n) is an integer from 1 to 7 and (m) is an integer from 1 to 3;
when R1 is methyl, ethyl or isopropyl, (n) is an integer from 1 to 7 and (m) is an integer from 1 to 2; and
wherein the oil viscosity is lower than 3.0 cSt at the temperature of 40° C.

3. An oil according to claim 1, characterized in that the oil viscosity is between 1.0 and 3.0 cSt at the temperature of 40° C.

4. An oil according to claim 1, characterized in that the oil viscosity is between 1.0 and below 2.5 cSt at the temperature of 40° C.

5. An oil according to claim 1, characterized in that the oil viscosity is between 1.0 and below 2.2 cSt at the temperature of 40° C.

6. An oil according to claim 1, characterized in that the total amount of hydrocarbon radicals bonded to the aromatic ring is between 1 and 12.

7. An oil according to claim 1, characterized in that at least 80% by mass of the oil consists of the at least one alkyl aromatic compound.

8. An oil according to claim 1, characterized in that it additionally comprises at least one additive selected from the group consisting of oxidation resistance enhancers, thermal stability enhancers, corrosion inhibitors, metal deactivators, lubricity additives, viscosity index enhancers, fluidity lowering agents, floc point lowering agents, detergents, dispersants, foaming agents, anti-wear agents and high-pressure resistance agents.

9. An oil according to claim 1, characterized in that the alkyl aromatic compound comprises a molecular weight between 134 and 218.

10. An oil according to claim 1, characterized in that it is used as a lubricating oil.

11. An oil according to claim 1, characterized in that the alkyl aromatic compound is a linear alkyl aromatic compound.

12. Lubricant composition, characterized in that it comprises:
at least 80% by mass of the oil as defined in claim 1;
a cooling fluid from the HC (hydrocarbon) group; and
at least one additive.

13. Lubricant composition according to claim 8, characterized in that the cooling fluid from the HC group is selected from HC-600a, HC-290 or combinations thereof.

14. Use of the oil as claimed in claim 1 characterized in that it is for manufacturing a mechanical equipment.

15. Use according to claim 14, characterized in that the mechanical equipment is a cooling machine.

16. Use according to claim 15, characterized in that the cooling machine is a compressor.

17. Use of the lubricant composition as claimed in claim 12, characterized in that it is for manufacturing cooling machines.

18. Use according to claim 17, characterized in that the cooling machines are compressors or hermetic compressors.

19. Mechanical equipment, characterized in that it comprises, an alkylaromatic oil, as defined in claim 1.

20. Mechanical equipment according to claim 19, characterized in that the mechanical equipment is a compressor, hermetic compressor or cooling machine.
